(19) United States
(12) Patent Application Publication
(10) Pub. No.: US 2003/0229321 A1
(43) Pub. Date: Dec. 11, 2003

(54) Needle with slotted tip

(76) Inventor: Timothy Simon, Los Alamitos, CA (US)

Correspondence Address: FULWIDER PATTON LEE & UTECHT, LLP, 200 OCEANGATE, SUITE 1550, LONG BEACH, CA, 90802, US

(21) Appl. No.: 10/163,741

(22) Filed: Jun. 5, 2002

(51) Int. Cl. A61M 5/32

(52) U.S. Cl. 604/272

(57) ABSTRACT

A manual surgical instrument needle for the injection of fluid into intraarticular space having a helical slot formed on its lateral surface and a closed off distal end. The slot ensures that a direct flow path into the intraarticular space is established despite the embedment of the distal tip of the needle into articular cartilage or bone or soft tissue and regardless of the needle's rotational orientation.

### BACKGROUND OF THE INVENTION

The present invention relates to a class of manual surgical instruments which include disposable or reusable aspiration and injection needles. For simplicity, the device of the present invention will be referred to as “needle” hereafter. The present invention relates to needles used for aspiration or injection of fluids and is more particularly directed to needles used for injecting fluid into an intraarticular space.

Osteoarthritis is one of the most common and costly chronic medical conditions. At present, most therapies are directed towards minimizing pain and swelling, maintaining joint mobility and reducing associated disability. Non-steroidal anti-inflammatory drugs are the most widely used medications and have been the mainstay of treatment by physicians and over the counter use by patients. Alternative therapies are however gaining in popularity.

In osteoarthritis, there is often a reduction in the elastoviscosity of the synovial joint fluid secondary to a decrease in the molecular weight and concentration of hyaluronic acid. Viscosupplementation is a therapeutic technique that addresses the decrease in synovial viscosity with the injection of high-molecular-weight hyaluronan molecules. Viscosupplementation was initially used to treat post-traumatic osteoarthritis in race horses, and later used for human knee arthritis in the early 1970's. Several human clinical trials have shown a single course of three weekly injections of hyaluronan was more effective than saline controls, and equivalent to or better than continuous non-steroidal anti-inflammatory drug therapy plus arthrocentesis.

Hyaluronan has been approved as an intraarticular device to coat the articular surfaces and synovial lining in the knee joint. However, in order to achieve maximal therapeutic benefit from hyaluronic acid derivative injections, the material must be delivered directly into the knee joint space as its high viscosity precludes its diffusion there into from the surrounding tissue. This is in contrast to intraarticular injections of for example cortisone wherein accurate placement is not as critical as its low viscosity allows it to readily diffuse and thereby achieve a clinical response.

Achieving accurate positioning of the distal end of an injection needle is difficult and studies have shown that clinicians are often unable to achieve proper intraarticular placement. Often, a clinician can only rely on effusion that may be present in the intraarticular space in order to confirm proper placement, whereby the ability to aspirate such fluid from the joint indicates that the needle tip is in fact positioned in the intrarticular space rather than proximally thereto in the fat pad or distally thereto, embedded in the cartilage or bone. The absence of effusion in the intraarticular space would of course preclude the use of such technique altogether and further compounds the problem as the intraarticular space is as a result much smaller. The injection of hyaluroran is therefore often less effective than it could be by virtue of the fact that it is simply not delivered to the appropriate place.

An additional problem associated with the use of the conventional hypodermic needles to gain access to the intraarticular space is that the hollow configuration of the distal end of the needle has a cookie-cutter effect and therefore has a tendency to detach a plug of synovial tissue which is then injected into the joint along with the hyaluronan. The presence of such debris within the intraarticular space has a deleterious effect and is therefore to be avoided.

An improved device is therefore needed with which the intraarticular space can readily be accessed with minimal effort and without the need to rely on effusion to confirm proper placement. Additionally, it is highly desirable to be able to access the intraarticular space without the risk of transferring detached particles of synovial tissue there into.

### SUMMARY OF THE INVENTION

The needle of the present invention overcomes the shortcomings of devices previously used for injecting fluids into intraarticular spaces. The use of the needle greatly simplifies the clinician's task and ensures that injectant reaches the intraarticular space without requiring insertion to a precise depth, precisely maintaining such depth and without the need to rely on the presence of effusion to confirm placement. Additionally, the needle of the present invention prevents the detachment of synovial tissue and the subsequent transfer of such tissue into the intraarticular space. Finally, the device of the present invention may optionally be configured to greatly improve its tracking ability to allow the clinician to more accurately control the path of the needle as it is advanced through tissue.

The needle of the present invention has a helical slot formed in its lateral surface. The slot extends from just proximal to the needle's tip along approximately 9.0-10.0 mm of its longitudinal length and subtends an angle of about 360°. The helical nature of the slot ensures that the interior of the needle is set into direct fluid communication with intraarticular space upon embedment of the needle tip in the articular cartilage and irrespective of its rotational orientation. The needle tip is sealed off in order to prevent the detachment and capture of tissue as the needle is advanced through the various layers of tissue. A temporarily inserted trochar may be used to keep tissue from entering the lumen of the needle and may enhance the strength of the needle during the insertion step. The needle may optionally be formed with a conical tip (a preferred embodiment) rather than with a beveled tip in order to enhance its trackability.

These and other features and advantages of the present invention will become apparent from the following detailed description of a preferred embodiment which, taken in conjunction with the accompanying drawings, illustrates by way of example the principles of the invention.

### BRIEF DESCRIPTION OF THE DRAWINGS

FIGS. 1a-c are side views of the needle of the present invention in successive rotational orientations;

FIG. 2 is a side view of an alternative embodiment of the needle of the present invention;

FIG. 3 is a side view of a trochar for use during the placement of the needle of the present invention; and

FIG. 4 is an enlarged cross-sectional view of a knee joint with the needle of the present invention inserted thereinto.

### DETAILED DESCRIPTION OF THE PREFERRED EMBODIMENTS

The needle of the present invention facilitates the injection of fluid into the intraarticular space. The device ensures the flow of fluid directly into the space despite the embedment of the needle tip in cartilage and/or despite only minimal spacing between the cartilage and the fat pad. Moreover, the reliance on effusion is not necessary for confirming the proper placement of the needle.

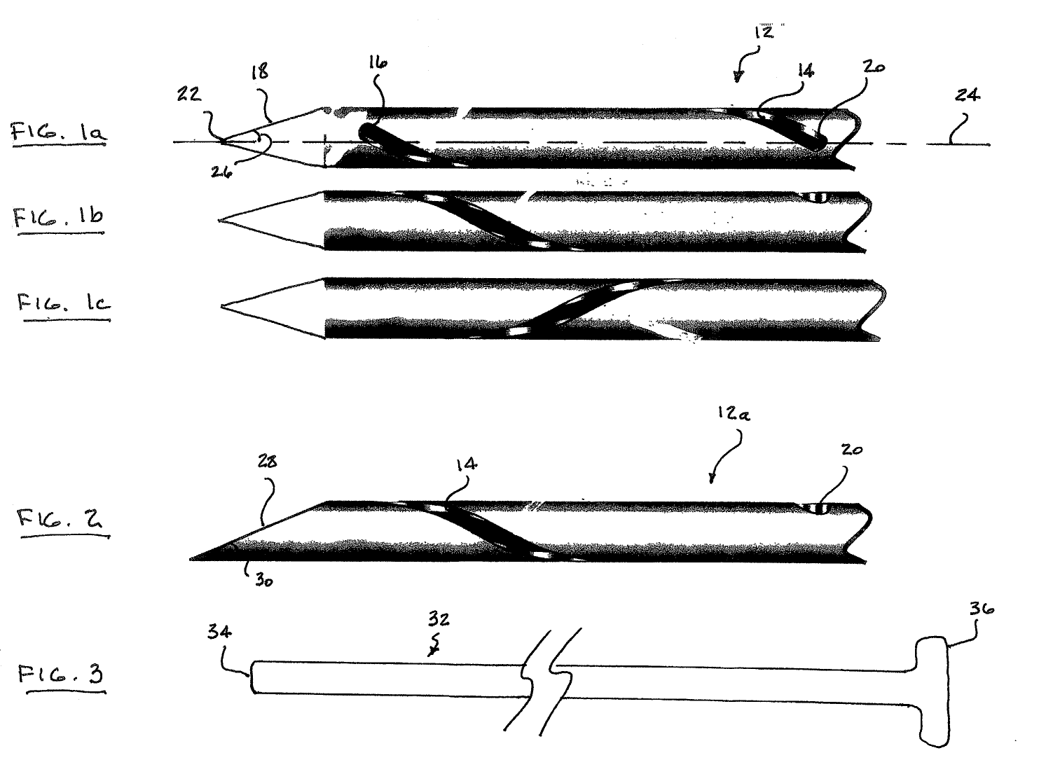

FIGS. 1a-c illustrate the needle 12 of the present invention which has a helical slot 14 formed in its lateral surface. The distal end 16 of the slot is located just proximal to the tapered portion of the needle's distal tip 18 while the proximal end 20 of the slot is distanced approximately 0.3 mm from its distal end 22 as measured along the needle's longitudinal axis 24. The slot spirals along the needle's lateral surface so as to subtend an angle of approximately 360°. The slot preferably has a constant width along its entire length of approximately 10.3 mm and has rounded ends 16, 20.

In the preferred embodiment illustrated in FIGS. 1a-c, the needle tip 18 is closed off and has a conical form wherein the distal end 22 of the tip is aligned with the needle's longitudinal axis 24. The outer surface of the conical tip preferably defines an angle 26 of approximately 12° relative to the needle's longitudinal axis.

FIG. 2 illustrates an alternative embodiment 12a of the needle of the present invention wherein a beveled tip 28 is formed on the distal end of the needle. The tip is closed off and the bevel defines an angle 30 of approximately 22°. Other embodiments of this tip angle are envisioned to optimize tissue penetration.

The dimensions of the needle of the present invention are dependent upon the specific application for which the needle is intended. For use in a human knee, an 18-22 gauge stainless steel hypodermic needle approximately 2-3.5 inches in length is preferred. The proximal end of the needle may be fitted with a standard luer lock for use with a standard syringe.

The slot 14 may be formed in the needle using any of various standard manufacturing methods. A preferred method calls for the use of laser cutting or EDM. Slash grinding has also been successfully employed. The distal end of beveled needle may closed off by a weld while a closed of conical end may be formed by pressure rolling or swedging the tip closed.

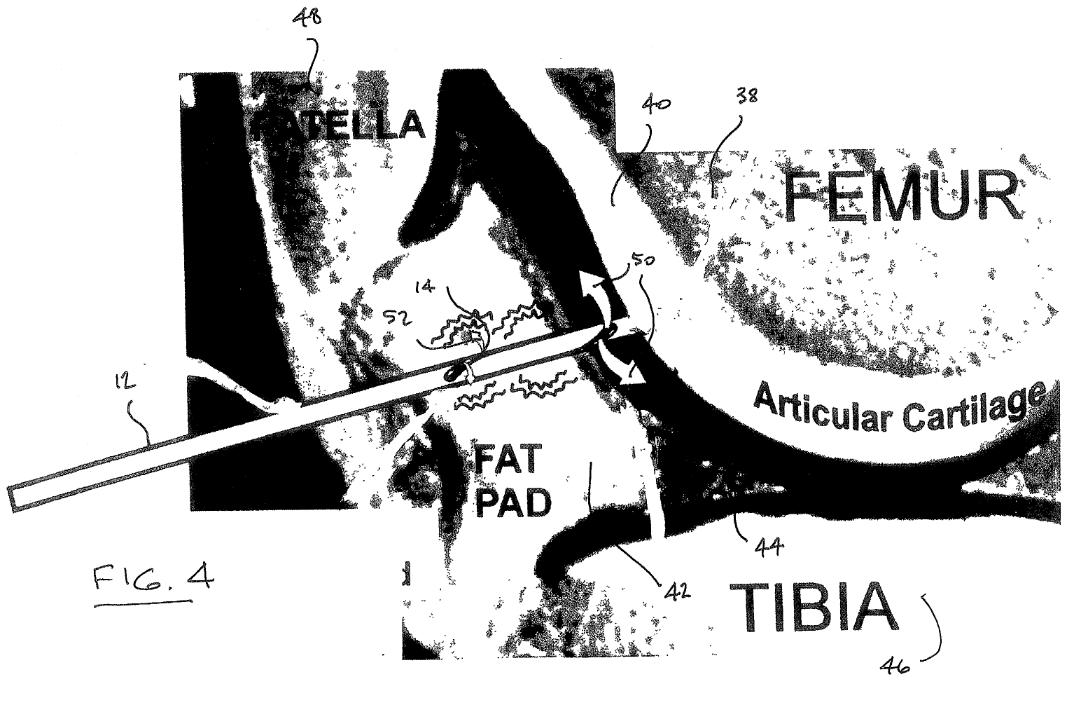

FIG. 3 illustrates a trochar 32 that may advantageously be used in combination with the needles illustrated in the Figures while the needle is being advanced through tissue into the joint. The outer diameter of the trochar is slightly less than the inner diameter of the needle while its distal end 34 may have a flat or rounded configuration. Its length must exceed the length of the needle and may have a manipulator element 36 fitted to its proximal to enable to exert a distally directed force thereon and to facilitate the retraction of the trochar after the needle has been placed.

FIG. 4 is a cross-sectional view of a human knee showing the femur 38, the articular cartilage 40 that lines the femur, the fat pad 42 and the intraarticular space 44 situated therebetween. The tibia 46 and patella 48 are also visible. In use, the needle 12 is inserted into the fat pad just below the patella (anteromedial portal) and advanced therethrough until it impacts the bony wall of the intercondylar notch or the articular cartilage. A trochar may be used to advance the needle into place in an effort to cause force to be exerted directly on the interior surface of the distal tip rather than being transferred thereto across the slotted section, and in addition, keeps soft tissue from entering the lumen of the needle. The use of the conical tip configuration as shown in FIGS. 1a-c rather than a beveled tip configuration as shown in FIG. 2 improves the tracking of the needle to thereby more accurately follow the path intended by the clinician. The fact that the distal end of the needle is closed off in either configuration prevents the detachment of tissue through which it is being advanced by a cookie-cutter effect and thereby prevents the subsequent injection of such tissue into the intraarticular space. The rounded shape of the proximal and distal ends of the slot 16, 20 similarly prevent the detachment of tissue as the needle is advanced therethrough. Once the needle is in place, the trochar is removed and a syringe is attached to the proximal end of the needle to facilitate the injection of viscous solutions or medication for example hyaluronan. The helical configuration of slot 14 guarantees that a direct flowpath 50 to the intraarticular space is established while the substantial backpressure created by the fat pad tissue density that surrounds the proximal portion of the slot prevents the escape of any substantial amount into the fat tissue (52). Similarly, back pressure of articular cartilage, ligamentous and/or connective tissue that surrounds the distal portion of the slot prevents the escape of any substantial amounts of fluid into these tissues.

While a particular form of the invention has been illustrated and described, it will also be apparent to those skilled in the art that various modifications can be made without departing from the spirit and scope of the invention. More particularly, the dimensions of the needle, including the width, length and positioning of the slot may be varied to accommodate different sized patients and different joints such as the shoulder or hip. Additionally, the needle can be used for injecting medicines such as those based on polymeric solutions for slow release or local release. Accordingly, it is not intended that the invention be limited except by the appended claims.

What is claimed is:

1. A device for injecting fluid, comprising a hollow needle with a lateral wall having a helical slot formed there through.

2. The device of claim 1, wherein said helical slot subtends an angle of about 360°.

3. The device of claim 1, wherein said hollow needle has a closed off distal end.

4. The device of claim 3, wherein said closed off distal end has a conical configuration.

5. The device of claim 3, wherein said closed off distal end has a beveled configuration.

6. The needle of claim 1, wherein said slot extends about 9.0-10.0 mm along a longitudinal axis defined by said needle.

7. The needle of claim 1, wherein said proximal end of said needle is fitted with a luer lock mechanism.

8. A device for injecting fluid into an intraarticular space, comprising a hollow needle with a lateral wall having a helical slot formed there through and a closed off tapered distal end, wherein said slot terminates at a point just proximal to said taper, subtends an angle of approximately 360° and extends along the needles longitudinal axis a distance of about 9.0-10.0 mm.

9. The device of claim 8, wherein said tapered end has a conical configuration.

10. The device of claim 8, wherein said tapered end has a beveled configuration.
